SEQUENCE LISTING (57 sequences)

SEQ ID NO: 1 - protein, 24 aa, synthetic construct
IGKEFKRIVERKKRFLRELVRPLR

SEQ ID NO: 2 - protein, 24 aa, synthetic construct
IGKEFKRIVERKWRFLRELVRPLR

SEQ ID NO: 3 - protein, 24 aa, synthetic construct
IGKKFKRIVRRKKRFLRKLVRPLR

SEQ ID NO: 4 - protein, 24 aa, synthetic construct
IGKEFKRIVERKWRFLRKLVRPLR

SEQ ID NO: 5 - protein, 24 aa, synthetic construct
IGKEFLRIVERKWRFLRKLVRPLL

SEQ ID NO: 6 - protein, 24 aa, synthetic construct
IGKEFLRIVERKWRFLVKLVRPLL

SEQ ID NO: 7 - protein, 24 aa, synthetic construct
  VARIANT 4: note = E, K, R, L, I, V, F, A, W, V, P
  VARIANT 6: note = E, K, R, L, I, V, F, A, W, V, P
  VARIANT 10: note = E, K, R, L, I, V, F, A, W, V, P
  VARIANT 13: note = E, K, R, L, I, V, F, A, W, V, P
  VARIANT 17: note = E, K, R, L, I, V, F, A, W, V, P
  VARIANT 18: note = E, K, R, L, I, V, F, A, W, V, P
  VARIANT 18: note = E, K, R, L, I, V, F, A, W, V, P
  VARIANT 24: note = E, K, R, L, I, V, F, A, W, V, P
IGKXFXRIVXRKXRFLXXLVRPLX

SEQ ID NO: 8 - protein, 24 aa, synthetic construct
IGKEFKRIVERKKRFLRELVRPLR

SEQ ID NO: 9 - protein, 24 aa, synthetic construct
IGKEFKRIVERKWRFLRELVRPLR

SEQ ID NO: 10 - protein, 24 aa, synthetic construct
IGKKFKRIVRRKKRFLRKLVRPLR

SEQ ID NO: 11 - protein, 24 aa, synthetic construct
IGKEFKRIVERKWRFLRKLVRPLR

SEQ ID NO: 12 - protein, 24 aa, synthetic construct
IGKEFLRIVERKWRFLRKLVRPLL

SEQ ID NO: 13 - protein, 24 aa, synthetic construct
IGKEFLRIVERKWRFLVKLVRPLL

SEQ ID NO: 14 - protein, 14 aa, synthetic construct
  VARIANT 1: note = E, K, R, L, I, V, F, A, W, V, P
  VARIANT 5: note = E, K, R, L, I, V, F, A, W, V, P
  VARIANT 8: note = E, K, R, L, I, V, F, A, W, V, P
  VARIANT 12: note = E, K, R, L, I, V, F, A, W, V, P
  VARIANT 13: note = E, K, R, L, I, V, F, A, W, V, P
XRIVXRKXRFLXXL

SEQ ID NO: 15 - protein, 14 aa, synthetic construct
  VARIANT 1: note = E, K, R, L, I, V, F, A, W, V, P
  VARIANT 5: note = E, K, R, L, I, V, F, A, W, V, P
  VARIANT 8: note = E, K, R, L, I, V, F, A, W, V, P
  VARIANT 12: note = E, K, R, L, I, V, F, A, W, V, P
  VARIANT 13: note = E, K, R, L, I, V, F, A, W, V, P
XRIVXRKXRFLXXL

SEQ ID NO: 16 - protein, 15 aa, synthetic construct
  VARIANT 1: note = E, K, R, L, I, V, F, A, W, V, P
  VARIANT 5: note = E, K, R, L, I, V, F, A, W, V, P
  VARIANT 12: note = E, K, R, L, I, V, F, A, W, V, P
  VARIANT 13: note = E, K, R, L, I, V, F, A, W, V, P
  VARIANT 8: note = E, K, R, L, I, V, F, A, W, V, P
XRIVXRKXRFLXXLV

SEQ ID NO: 17 - protein, 16 aa, synthetic construct
  VARIANT 1: note = E, K, R, L, I, V, F, A, W, V, P
  VARIANT 5: note = E, K, R, L, I, V, F, A, W, V, P
  VARIANT 8: note = E, K, R, L, I, V, F, A, W, V, P
  VARIANT 12: note = E, K, R, L, I, V, F, A, W, V, P
  VARIANT 13: note = E, K, R, L, I, V, F, A, W, V, P
XRIVXRKXRFLXXLVR

SEQ ID NO: 18 - protein, 17 aa, synthetic construct
  VARIANT 1: note = E, K, R, L, I, V, F, A, W, V, P
  VARIANT 5: note = E, K, R, L, I, V, F, A, W, V, P
  VARIANT 8: note = E, K, R, L, I, V, F, A, W, V, P
  VARIANT 12: note = E, K, R, L, I, V, F, A, W, V, P
  VARIANT 13: note = E, K, R, L, I, V, F, A, W, V, P
XRIVXRKXRFLXXLVRP

SEQ ID NO: 19 - protein, 18 aa, synthetic construct
  VARIANT 1: note = E, K, R, L, I, V, F, A, W, V, P
  VARIANT 5: note = E, K, R, L, I, V, F, A, W, V, P
  VARIANT 8: note = E, K, R, L, I, V, F, A, W, V, P
  VARIANT 12: note = E, K, R, L, I, V, F, A, W, V, P
  VARIANT 13: note = E, K, R, L, I, V, F, A, W, V, P
XRIVXRKXRFLXXLVRPL

SEQ ID NO: 20 - protein, 19 aa, synthetic construct
  VARIANT 1: note = E, K, R, L, I, V, F, A, W, V, P
  VARIANT 5: note = E, K, R, L, I, V, F, A, W, V, P
  VARIANT 8: note = E, K, R, L, I, V, F, A, W, V, P
  VARIANT 12: note = E, K, R, L, I, V, F, A, W, V, P
  VARIANT 13: note = E, K, R, L, I, V, F, A, W, V, P
XRIVXRKXRFLXXLVRPLR

SEQ ID NO: 21 - protein, 15 aa, synthetic construct
  VARIANT 2: note = E, K, R, L, I, V, F, A, W, V, P
  VARIANT 6: note = E, K, R, L, I, V, F, A, W, V, P
  VARIANT 9: note = E, K, R, L, I, V, F, A, W, V, P
  VARIANT 13: note = E, K, R, L, I, V, F, A, W, V, P
  VARIANT 14: note = E, K, R, L, I, V, F, A, W, V, P
FXRIVXRKXRFLXXL

SEQ ID NO: 22 - protein, 16 aa, synthetic construct
  VARIANT 2: note = E, K, R, L, I, V, F, A, W, V, P
  VARIANT 6: note = E, K, R, L, I, V, F, A, W, V, P
  VARIANT 9: note = E, K, R, L, I, V, F, A, W, V, P
  VARIANT 13: note = E, K, R, L, I, V, F, A, W, V, P
  VARIANT 14: note = 14
FXRIVXRKXRFLXXLV

SEQ ID NO: 23 - protein, 17 aa, synthetic construct
  VARIANT 2: note = E, K, R, L, I, V, F, A, W, V, P
  VARIANT 6: note = E, K, R, L, I, V, F, A, W, V, P
  VARIANT 9: note = E, K, R, L, I, V, F, A, W, V, P
  VARIANT 13: note = E, K, R, L, I, V, F, A, W, V, P
  VARIANT 14: note = E, K, R, L, I, V, F, A, W, V, P
FXRIVXRKXRFLXXLVR

SEQ ID NO: 24 - protein, 18 aa, synthetic construct
  VARIANT 2: note = E, K, R, L, I, V, F, A, W, V, P
  VARIANT 6: note = E, K, R, L, I, V, F, A, W, V, P
  VARIANT 9: note = E, K, R, L, I, V, F, A, W, V, P
  VARIANT 13: note = E, K, R, L, I, V, F, A, W, V, P
  VARIANT 14: note = E, K, R, L, I, V, F, A, W, V, P
FXRIVXRKXRFLXXLVRP

SEQ ID NO: 25 - protein, 19 aa, synthetic construct
  VARIANT 2: note = E, K, R, L, I, V, F, A, W, V, P
  VARIANT 6: note = E, K, R, L, I, V, F, A, W, V, P
  VARIANT 9: note = E, K, R, L, I, V, F, A, W, V, P
  VARIANT 13: note = E, K, R, L, I, V, F, A, W, V, P
  VARIANT 14: note = E, K, R, L, I, V, F, A, W, V, P
FXRIVXRKXRFLXXLVRPL

SEQ ID NO: 26 - protein, 20 aa, synthetic construct
  VARIANT 2: note = E, K, R, L, I, V, F, A, W, V, P
  VARIANT 6: note = E, K, R, L, I, V, F, A, W, V, P
  VARIANT 9: note = E, K, R, L, I, V, F, A, W, V, P
  VARIANT 13: note = E, K, R, L, I, V, F, A, W, V, P
  VARIANT 14: note = E, K, R, L, I, V, F, A, W, V, P
FXRIVXRKXRFLXXLVRPLR

SEQ ID NO: 27 - protein, 16 aa, synthetic construct
  VARIANT 3: note = E, K, R, L, I, V, F, A, W, V, P
  VARIANT 7: note = E, K, R, L, I, V, F, A, W, V, P
  VARIANT 10: note = E, K, R, L, I, V, F, A, W, V, P
  VARIANT 14: note = E, K, R, L, I, V, F, A, W, V, P
  VARIANT 15: note = E, K, R, L, I, V, F, A, W, V, P
EFXRIVXRKXRFLXXL

SEQ ID NO: 28 - protein, 17 aa, synthetic construct
  VARIANT 3: note = E, K, R, L, I, V, F, A, W, V, P
  VARIANT 7: note = E, K, R, L, I, V, F, A, W, V, P
  VARIANT 10: note = E, K, R, L, I, V, F, A, W, V, P
  VARIANT 14: note = E, K, R, L, I, V, F, A, W, V, P
  VARIANT 15: note = E, K, R, L, I, V, F, A, W, V, P
EFXRIVXRKXRFLXXLV

SEQ ID NO: 29 - protein, 18 aa, synthetic construct
  VARIANT 3: note = E, K, R, L, I, V, F, A, W, V, P
  VARIANT 7: note = E, K, R, L, I, V, F, A, W, V, P
  VARIANT 10: note = E, K, R, L, I, V, F, A, W, V, P
  VARIANT 14: note = E, K, R, L, I, V, F, A, W, V, P
  VARIANT 15: note = E, K, R, L, I, V, F, A, W, V, P
EFXRIVXRKXRFLXXLVR

SEQ ID NO: 30 - protein, 19 aa, synthetic construct
  VARIANT 3: note = E, K, R, L, I, V, F, A, W, V, P
  VARIANT 7: note = E, K, R, L, I, V, F, A, W, V, P
  VARIANT 10: note = E, K, R, L, I, V, F, A, W, V, P
  VARIANT 14: note = E, K, R, L, I, V, F, A, W, V, P
  VARIANT 15: note = E, K, R, L, I, V, F, A, W, V, P
EFXRIVXRKXRFLXXLVRP

SEQ ID NO: 31 - protein, 20 aa, synthetic construct
  VARIANT 3: note = E, K, R, L, I, V, F, A, W, V, P
  VARIANT 7: note = E, K, R, L, I, V, F, A, W, V, P
  VARIANT 10: note = E, K, R, L, I, V, F, A, W, V, P
  VARIANT 14: note = E, K, R, L, I, V, F, A, W, V, P
  VARIANT 15: note = E, K, R, L, I, V, F, A, W, V, P
EFXRIVXRKXRFLXXLVRPL

SEQ ID NO: 32 - protein, 21 aa, synthetic construct
  VARIANT 3: note = E, K, R, L, I, V, F, A, W, V, P
  VARIANT 7: note = E, K, R, L, I, V, F, A, W, V, P
  VARIANT 10: note = E, K, R, L, I, V, F, A, W, V, P
  VARIANT 14: note = E, K, R, L, I, V, F, A, W, V, P
  VARIANT 15: note = E, K, R, L, I, V, F, A, W, V, P
EFXRIVXRKXRFLXXLVRPLR

SEQ ID NO: 33 - protein, 17 aa, synthetic construct
  VARIANT 4: note = E, K, R, L, I, V, F, A, W, V, P
  VARIANT 8: note = E, K, R, L, I, V, F, A, W, V, P
  VARIANT 11: note = E, K, R, L, I, V, F, A, W, V, P
  VARIANT 15: note = E, K, R, L, I, V, F, A, W, V, P
  VARIANT 16: note = E, K, R, L, I, V, F, A, W, V, P
KEFXRIVXRKXRFLXXL

SEQ ID NO: 34 - protein, 18 aa, synthetic construct
  VARIANT 4: note = E, K, R, L, I, V, F, A, W, V, P
  VARIANT 8: note = E, K, R, L, I, V, F, A, W, V, P
  VARIANT 11: note = E, K, R, L, I, V, F, A, W, V, P
  VARIANT 15: note = E, K, R, L, I, V, F, A, W, V, P
  VARIANT 16: note = E, K, R, L, I, V, F, A, W, V, P
KEFXRIVXRKXRFLXXLV

SEQ ID NO: 35 - protein, 19 aa, synthetic construct
  VARIANT 4: note = E, K, R, L, I, V, F, A, W, V, P
  VARIANT 8: note = E, K, R, L, I, V, F, A, W, V, P
  VARIANT 11: note = E, K, R, L, I, V, F, A, W, V, P
  VARIANT 15: note = E, K, R, L, I, V, F, A, W, V, P
  VARIANT 16: note = E, K, R, L, I, V, F, A, W, V, P
KEFXRIVXRKXRFLXXLVR

SEQ ID NO: 36 - protein, 20 aa, synthetic construct
  VARIANT 4: note = E, K, R, L, I, V, F, A, W, V, P
  VARIANT 8: note = E, K, R, L, I, V, F, A, W, V, P
  VARIANT 11: note = E, K, R, L, I, V, F, A, W, V, P
  VARIANT 15: note = E, K, R, L, I, V, F, A, W, V, P
  VARIANT 16: note = E, K, R, L, I, V, F, A, W, V, P
KEFXRIVXRKXRFLXXLVRP

SEQ ID NO: 37 - protein, 21 aa, synthetic construct
  VARIANT 4: note = E, K, R, L, I, V, F, A, W, V, P
  VARIANT 8: note = E, K, R, L, I, V, F, A, W, V, P
  VARIANT 11: note = E, K, R, L, I, V, F, A, W, V, P
  VARIANT 15: note = E, K, R, L, I, V, F, A, W, V, P
  VARIANT 16: note = E, K, R, L, I, V, F, A, W, V, P
KEFXRIVXRKXRFLXXLVRPL

SEQ ID NO: 38 - protein, 22 aa, synthetic construct
  VARIANT 4: note = E, K, R, L, I, V, F, A, W, V, P
  VARIANT 8: note = E, K, R, L, I, V, F, A, W, V, P
  VARIANT 11: note = E, K, R, L, I, V, F, A, W, V, P
  VARIANT 15: note = E, K, R, L, I, V, F, A, W, V, P
  VARIANT 16: note = E, K, R, L, I, V, F, A, W, V, P
KEFXRIVXRKXRFLXXLVRPLR

SEQ ID NO: 39 - protein, 18 aa, synthetic construct
  VARIANT 5: note = E, K, R, L, I, V, F, A, W, V, P
  VARIANT 9: note = E, K, R, L, I, V, F, A, W, V, P
  VARIANT 12: note = E, K, R, L, I, V, F, A, W, V, P
  VARIANT 16: note = E, K, R, L, I, V, F, A, W, V, P
  VARIANT 17: note = E, K, R, L, I, V, F, A, W, V, P
GKEFXRIVXRKXRFLXXL

SEQ ID NO: 40 - protein, 19 aa, synthetic construct
  VARIANT 5: note = E, K, R, L, I, V, F, A, W, V, P
  VARIANT 9: note = E, K, R, L, I, V, F, A, W, V, P
  VARIANT 12: note = E, K, R, L, I, V, F, A, W, V, P
  VARIANT 16: note = E, K, R, L, I, V, F, A, W, V, P
  VARIANT 17: note = E, K, R, L, I, V, F, A, W, V, P
GKEFXRIVXRKXRFLXXLV

SEQ ID NO: 41 - protein, 20 aa, synthetic construct
  VARIANT 5: note = E, K, R, L, I, V, F, A, W, V, P
  VARIANT 9: note = E, K, R, L, I, V, F, A, W, V, P
  VARIANT 12: note = E, K, R, L, I, V, F, A, W, V, P
  VARIANT 16: note = E, K, R, L, I, V, F, A, W, V, P
  VARIANT 17: note = E, K, R, L, I, V, F, A, W, V, P
GKEFXRIVXRKXRFLXXLVR

SEQ ID NO: 42 - protein, 21 aa, synthetic construct
  VARIANT 5: note = E, K, R, L, I, V, F, A, W, V, P
  VARIANT 9: note = E, K, R, L, I, V, F, A, W, V, P
  VARIANT 12: note = E, K, R, L, I, V, F, A, W, V, P
  VARIANT 16: note = E, K, R, L, I, V, F, A, W, V, P
  VARIANT 17: note = E, K, R, L, I, V, F, A, W, V, P
GKEFXRIVXRKXRFLXXLVRP

SEQ ID NO: 43 - protein, 22 aa, synthetic construct
  VARIANT 5: note = E, K, R, L, I, V, F, A, W, V, P
  VARIANT 9: note = E, K, R, L, I, V, F, A, W, V, P
  VARIANT 12: note = E, K, R, L, I, V, F, A, W, V, P
  VARIANT 16: note = E, K, R, L, I, V, F, A, W, V, P
  VARIANT 17: note = E, K, R, L, I, V, F, A, W, V, P
GKEFXRIVXRKXRFLXXLVRPL

SEQ ID NO: 44 - protein, 23 aa, synthetic construct
  VARIANT 5: note = E, K, R, L, I, V, F, A, W, V, P
  VARIANT 9: note = E, K, R, L, I, V, F, A, W, V, P
  VARIANT 12: note = E, K, R, L, I, V, F, A, W, V, P
  VARIANT 16: note = E, K, R, L, I, V, F, A, W, V, P
  VARIANT 17: note = E, K, R, L, I, V, F, A, W, V, P
GKEFXRIVXRKXRFLXXLVRPLR

SEQ ID NO: 45 - protein, 19 aa, synthetic construct
  VARIANT 6: note = E, K, R, L, I, V, F, A, W, V, P
  VARIANT 10: note = E, K, R, L, I, V, F, A, W, V, P
  VARIANT 13: note = E, K, R, L, I, V, F, A, W, V, P
  VARIANT 17: note = E, K, R, L, I, V, F, A, W, V, P
  VARIANT 18: note = E, K, R, L, I, V, F, A, W, V, P
IGKEFXRIVXRKXRFLXXL

SEQ ID NO: 46 - protein, 20 aa, synthetic construct
  VARIANT 6: note = E, K, R, L, I, V, F, A, W, V, P
  VARIANT 10: note = E, K, R, L, I, V, F, A, W, V, P
  VARIANT 13: note = E, K, R, L, I, V, F, A, W, V, P
  VARIANT 17: note = E, K, R, L, I, V, F, A, W, V, P
  VARIANT 18: note = E, K, R, L, I, V, F, A, W, V, P
IGKEFXRIVXRKXRFLXXLV

SEQ ID NO: 47 - protein, 21 aa, synthetic construct
  VARIANT 6: note = E, K, R, L, I, V, F, A, W, V, P
  VARIANT 10: note = E, K, R, L, I, V, F, A, W, V, P
  VARIANT 13: note = E, K, R, L, I, V, F, A, W, V, P
  VARIANT 17: note = E, K, R, L, I, V, F, A, W, V, P
  VARIANT 18: note = E, K, R, L, I, V, F, A, W, V, P
IGKEFXRIVXRKXRFLXXLVR

SEQ ID NO: 48 - protein, 22 aa, synthetic construct
  VARIANT 6: note = E, K, R, L, I, V, F, A, W, V, P
  VARIANT 10: note = E, K, R, L, I, V, F, A, W, V, P
  VARIANT 13: note = E, K, R, L, I, V, F, A, W, V, P
  VARIANT 17: note = E, K, R, L, I, V, F, A, W, V, P
  VARIANT 18: note = E, K, R, L, I, V, F, A, W, V, P
IGKEFXRIVXRKXRFLXXLVRP

SEQ ID NO: 49 - protein, 23 aa, synthetic construct
  VARIANT 6: note = E, K, R, L, I, V, F, A, W, V, P
  VARIANT 10: note = E, K, R, L, I, V, F, A, W, V, P
  VARIANT 13: note = E, K, R, L, I, V, F, A, W, V, P
  VARIANT 17: note = E, K, R, L, I, V, F, A, W, V, P
  VARIANT 18: note = E, K, R, L, I, V, F, A, W, V, P
IGKEFXRIVXRKXRFLXXLVRPL

SEQ ID NO: 50 - protein, 24 aa, synthetic construct
IGKEFKRIVERIKRFLRELVRPLR

SEQ ID NO: 51 - protein, 12 aa, synthetic construct
  VARIANT 1: note = E, K, R, L, I, V, F, A, W, V, P
  VARIANT 5: note = E, K, R, L, I, V, F, A, W, V, P
  VARIANT 8: note = E, K, R, L, I, V, F, A, W, V, P
  VARIANT 12: note = E, K, R, L, I, V, F, A, W, V, P
XRIVXRKXRFLX

SEQ ID NO: 52 - protein, 14 aa, synthetic construct
  VARIANT 1: note = E, K, R, L, I, V, F, A, W, V, P
  VARIANT 5: note = E, K, R, L, I, V, F, A, W, V, P
  VARIANT 8: note = E, K, R, L, I, V, F, A, W, V, P
  VARIANT 12: note = E, K, R, L, I, V, F, A, W, V, P
  VARIANT 13: note = E, K, R, L, I, V, F, A, W, V, P
XRIVXRKXRFLXXL

SEQ ID NO: 53 - protein, 14 aa, synthetic construct
KRIVERKWRFLREL

SEQ ID NO: 54 - protein, 14 aa, synthetic construct
KRIVRRKKRFLRKL

SEQ ID NO: 55 - protein, 14 aa, synthetic construct
KRIVERKWRFLRKL

SEQ ID NO: 56 - protein, 14 aa, synthetic construct
LRIVERKWRFLRKL

SEQ ID NO: 57 - protein, 14 aa, synthetic construct
LRIVERKWRFLVKL

(19) United States
(12) Patent Application Publication
(10) Pub. No.: US 2026/0285909 A1
(43) Pub. Date: Sep. 24, 2026

(54) PEPTIDES FOR ANTIMICROBIAL THERAPY

(71) Applicant: Karl-Franzens-Universität Graz, Graz (AT)

(72) Inventor: Nermina MALANOVIC, Graz (AT)

(21) Appl. No.: 19/477,378

(22) PCT Filed: Apr. 26, 2024
(86) PCT No.: PCT/EP2024/061649

§ 371 Date: Oct. 21, 2025

(30) Foreign Application Priority Data
Apr. 26, 2023 (AT) A 50313/2023

(51) Int. Cl. C07K 14/00; A61K 38/00; A61K 45/06; A61P 31/04

(52) U.S. Cl. CPC C07K 14/001; A61K 45/06; A61P 31/04; A61K 38/00

(57) ABSTRACT

Disclosed is an antimicrobial peptide comprising the sequence IGKX₁FX₂RIVX₃RKX₄RFLX₅X₆LVRPLX₇ (SEQ ID NO: 7), wherein each of X₁, X₂, X₃, X₄, X₅, X₆, and X₇ is an amino acid independently selected from the group consisting of E, K, R, L, I, V, F, A, W, V and P, preferably from the group consisting of E, K, R, L, I, V, F, A, W and V, more preferably from the group consisting of E, K, R, L, W and V. Optionally, one amino acid in this sequence selected from the group of L, V, F, A, I, W, Y or Q, with the exception of X₁-X₇, is replaced by another amino acid selected from said group or by P. Particularly preferred peptides are IGKEFKRIVERKWRFLRELVRPLR (SEQ ID NO: 2), IGKKFKRIVRRKKRFLRKLVRPLR (SEQ ID NO: 3), IGKEFKRIVERKWRFLRKLVRPLR (SEQ ID NO: 4), IGKEFLRIVERKWRFLRKLVRPLL (SEQ ID NO: 5) and IGKEFLRIVERKWRFLVKLVRPLL (SEQ ID NO: 6).

The field of the present invention relates to antimicrobial peptides.

Antimicrobial resistance has become a significant global health threat in recent years. The widespread overuse and misuse of antibiotics in medicine and agriculture have led to the emergence and rapid spread of antibiotic-resistant pathogens, rendering many conventional antibiotics less effective. Consequently, infections caused by antibiotic-resistant bacteria have resulted in increased morbidity and mortality, thereby increasing healthcare costs worldwide.

The World Health Organization regards antibiotic resistance as a top threat to global health. The increasing prevalence of multi-drug resistant bacteria has made many infections difficult or even impossible to treat with conventional antibiotics, underscoring the need for novel and effective antimicrobial agents to combat this global health crisis.

Against this background, host defense peptides have emerged as a promising alternative to traditional antibiotics. Host defense peptides are typically small and can be found in a wide range of organisms (e.g. in mammals, insects, amphibians). They play a crucial role in innate immunity.

Many host defense peptides can exhibit broad-spectrum antimicrobial activity against a wide range of pathogens, including bacteria, fungi, viruses, and parasites. Their multifaceted mode of action can involve not only direct antimicrobial effects through membrane disruption but also immunomodulatory activities, such as promoting wound healing and modulating inflammatory responses. Additionally, some host defense peptides have been shown to possess a lower propensity for inducing resistance compared to traditional antibiotics, making them particularly attractive as a basis for developing novel antimicrobial peptides (which may be derived from host defense peptides and harbor mutations e.g. for increasing their efficacy or reducing toxicity).

Examples for such peptides (and antimicrobial peptides derived therefrom) are disclosed in WO 2004/067563 A1 and WO 2008/002165 A1, as well as in Chen et al, 2021. Further examples are disclosed in WO 2014/182172 A1 and WO 2015/088344 A1.

Although many host defense peptides (and antimicrobial peptides derived therefrom) are known, there is a constant need for further antimicrobial peptides, in particular peptides which exhibit enhanced effectiveness against specific pathogens and/or possess a more favorable side effect profile, in particular in terms of reduced toxicity. Furthermore, while generally less prone to inducing resistance in microorganisms, eventual development of resistance remains a concern. Also therefore, it is beneficial to find additional antimicrobial peptides to provide additional options for antimicrobial therapy, in particular as second- or third-line treatment options.

It is thus an object of the present invention to provide antimicrobial peptides derived from host defense peptides, in particular with improved efficacy against certain microorganisms and/or with an improved side effect profile (in particular reduced toxicity).

The present invention provides an antimicrobial peptide comprising the sequence IGKX₁FX₂RIVX₃RKX₄RFLX₅X₆LVRPLX₇ (SEQ ID NO: 7), wherein each of X₁, X₂, X₃, X₄, X₅, X₆, and X₇ is an amino acid independently selected from the group consisting of E, K, R, L, I, V, F, A, W, V and P, preferably from the group consisting of E, K, R, L, I, V, F, A, W and V, more preferably from the group consisting of E, R, L, W and V (preferably with the proviso that the sequence is not IGKEFKRIVERKKRFLRELVRPLR (SEQ ID NO: 8)). Optionally, one further amino acid in the sequence IGKX₁FX₂RIVX₃RKX₄RFLX₅X₆LVRPLX₇ (SEQ ID NO: 7) selected from the group of L, V, F, A, I, W, Y or Q (with the exception of X₁-X₇) is replaced by another amino acid selected from said group or by P (preferably also with the proviso that the sequence is not IGKEFKRIVERKKRFLRELVRPLR (SEQ ID NO: 8)). Especially preferred is any one of the following sequences: IGKEFKRIVERKWRFLRELVRPLR (SEQ ID NO: 2), IGKKFKRIVRRKKRFLRKLVRPLR (SEQ ID NO: 3), IGKEFKRIVERKWRFLRKLVRPLR (SEQ ID NO: 4), IGKEFLRIVERKWRFLRKLVRPLL (SEQ ID NO: 5) and IGKEFLRIVERKWRFLVKLVRPLL (SEQ ID NO: 6).

The above-mentioned peptides may have a length of up to about 100 amino acids. However, shorter lengths are preferred, preferably less than 75 amino acids, more preferably less than 50 amino acids, even more preferably less than 40 amino acids, yet even more preferably less than 35 amino acids, especially less than 30 amino acids or even less than 27 amino acids. According to a particular preference, the entire amino acid sequence of the peptide consists of said amino-acid sequence or said variant of the amino acid sequence.

Furthermore, the present invention provides a peptide which is a fragment of the above-mentioned peptides. This fragment has the sequence X₂RIVX₃RKX₄RFLX₅X₆L (SEQ ID NO: 14), wherein each of X₂, X₃, X₄, X₅ and X₆ is an amino acid independently selected from the group consisting of E, K, R, L, I, V, F, A, W, V and P, preferably from the group consisting of E, K, R, L, I, V, F, A, W and V, more preferably from the group consisting of E, K, R, L, W and V; optionally wherein one amino acid in the sequence X₂RIVX₃RKX₄RFLX₅X₆L (SEQ ID NO: 14) selected from the group of L, V, F, A, I, W, Y or Q, with the exception of X₂-X₆, is replaced by another amino acid selected from said group or by P.

According to further embodiments, the length of the fragment peptide is 12-23 amino acids, preferably 13-22 amino acids or even 14-21 amino acids, more preferably 14-20 amino acids or even 14-19 amino acids, even more preferably 14-18 amino acids or even 14-17 amino acids, yet even more preferably 14-16 amino acids or even 14-15 amino acids, especially 14 amino acids.

Another aspect of the present invention relates to a method (for the prevention or treatment of a microbial infection), comprising obtaining a pharmaceutical composition comprising the peptide as defined herein, and administering the pharmaceutical composition to an individual, wherein the individual has a microbial infection or is at risk of developing a microbial infection.

Short antimicrobial peptide OP-145 (acetyl-IGKEFKRIVERIKRFLRELVRPLR-amide (SEQ ID NO: 50); also called P60.4Ac) is derived from the human cathelicidin LL-37 (see Haisma et al., 2014, and de Breij et al., 2016). In the course of the present invention, it was found that peptides derived from OP-145 with an I12K mutation (OP-145_{I12K}, SEQ ID NO: 1) and further mutations (having the general sequence IGKX₁FX₂RIVX₃RKX₄RFLX₅X₆LVRPLX₇ (SEQ ID NO: 7) as described herein), as well as fragments thereof, surprisingly exhibited high bactericidal activity against both Gram-negative and Gram-negative bacteria. This makes them exceptionally well suited as antimicrobial peptides.

The sequence of OP-145_{I12K} itself was published in Ön, 2017, and Piller, 2019. Unrelated to OP-145_{I12K}, Nell et al, 2006, discusses the development of novel LL-37-derived antimicrobial peptides with lipopolysaccharide (LPS)- and lipoteichoic acid (LTA)-neutralizing and antimicrobial activities for therapeutic application.

Two important parameters reflect the safety and efficacy of antimicrobial peptides: the lethal concentration (LC; see further below) and the hemolytic concentration (HC). The U.S. Food and Drug Administration (FDA) generally considers an antimicrobial peptide acceptable for therapeutic use if it exhibits a low level of hemolysis, typically no more than 5%, at the therapeutic concentrations. Hemolysis refers to the disruption of red blood cells, which can lead to the release of hemoglobin and other cellular contents into the bloodstream, potentially causing adverse effects.

More specifically, the HC refers to the lowest concentration of the peptide that induces a specific percentage of hemolysis, usually 5% or more, in a sample of red blood cells. The HC is generally determined through in vitro assays, where the peptide is exposed to red blood cells under controlled conditions, and the extent of hemolysis is quantified by measuring the release of hemoglobin or other cellular contents.

The therapeutic index (TI) is another key parameter that reflects safety and efficacy of antimicrobial peptides. The TI is calculated as the ratio of the HC to the LC (TI=HC/LC). A higher TI indicates a wider safety margin, as it implies that the peptide can achieve its antimicrobial activity at concentrations that are less likely to cause significant hemolysis (or other toxic effects).

Antimicrobial peptides of the present invention furthermore turned out to have a comparatively low hemolytic activity, leading to a higher therapeutic index.

Accordingly, the peptide (or fragment) preferably has a hemolytic activity which is lower than the hemolytic activity of peptide OP-145 (acetyl-IGKEFKRIVERIKRFLRELVRPLR-amide (SEQ ID NO: 50)), preferably at least 10% lower, more preferably at least 20% lower, even more preferably at least 30% lower, yet even more preferably at least 40% lower, especially at least 50% lower (e.g. at 1 μM or 10 μM or 20 μM or 50 μM).

In another preferred embodiment, the peptide (or fragment) has an antimicrobial activity which is higher than the antimicrobial activity of peptide OP-145 (acetyl-IGKEFKRIVERIKRFLRELVRPLR-amide (SEQ ID NO: 50)), preferably at least 10% higher, more preferably at least 20% higher, even more preferably at least 30% higher, yet even more preferably at least 40% higher, especially at least 50% higher.

In the course of the present invention, a consensus sequence was found which is particularly suitable for antimicrobial peptides. As mentioned above, this consensus sequence is IGKX₁FX₂RIVX₃RKX₄RFLX₅X₆LVRPLX₇ (SEQ ID NO: 7). The peptide of the present invention preferably comprises this sequence, with X₁ to X₇ being further defined as above. Optionally, one amino acid in the sequence IGKX₁FX₂RIVX₃RKX₄RFLX₅X₆LVRPLX₇ (SEQ ID NO: 7) selected from the group of L, V, F, A, I, W, Y or Q, with the exception of X₁-X₇, is replaced by another amino acid selected from said group or by P.

According to a particular preference, X₁ to X₇ are further defined as follows:
X₁ is E, K or R;
X₂ is E, K, R, L, I, V, F, A, W, or V;
X₃ is E, K or R;
X₄ is E, R, L, I, V, F, A, W, or V;
X₅ is E, K, R, L, I, V, F, A, W, or V;
X₆ is E, K or R; and
X₇ is E, K, R, L, I, V, F, A, W, or V.

More specifically, X₁ to X₇ are preferably defined as follows:
X₁ is E, K or R;
X₂ is K, L, I or V;
X₃ is E, K or R;
X₄ is L, I, V, F, A, W, or V;
X₅ is R, L, I or V;
X₆ is E, K or R; and
X₇ is R, L, I or V.

Even more specifically, X₁ to X₇ are preferably defined as follows:
X₁ is E or K;
X₂ is K or L;
X₃ is E or R;
X₄ is W;
X₅ is R or V;
X₆ is E or K; and
X₇ is R or L.

According to another particular preference, X₁ to X₇ are further defined as follows:
X₁ is K or R;
X₂ is E, K, R, L, I, V, F, A, W, or V;
X₃ is E, K or R;
X₄ is E, K, R, L, I, V, F, A, W, or V, preferably E, K or R, more preferably K;
X₅ is E, K, R, L, I, V, F, A, W, or V;
X₆ is E, K or R; and
X₇ is E, K, R, L, I, V, F, A, W, or V.

According to another particular preference, X₁ to X₇ are further defined as follows:
X₁ is E, K or R;
X₂ is E, K, R, L, I, V, F, A, W, or V;
X₃ is K or R;
X₄ is E, K, R, L, I, V, F, A, W, or V, preferably E, K or R, more preferably K;
X₅ is E, K, R, L, I, V, F, A, W, or V;
X₆ is E, K or R; and
X₇ is E, K, R, L, I, V, F, A, W, or V.

According to another particular preference, X₁ to X₇ are further defined as follows:
X₁ is E, K or R;
X₂ is E, K, R, L, I, V, F, A, W, or V;
X₃ is E, K or R;
X₄ is E, K, R, L, I, V, F, A, W, or V, preferably E, K or R, more preferably K;
X₅ is K, R, L, I, V, F, A, W, or V;
X₆ is E, K or R; and
X₇ is E, K, R, L, I, V, F, A, W, or V.

The consensus sequence is also suitable for the inventive fragment peptides. Preferably, the fragment of the fragment peptide is selected from:

(SEQ ID NO: 15)
X₂RIVX₃RKX₄RFLX₅X₆L,
(SEQ ID NO: 16)
X₂RIVX₃RKX₄RFLX₅X₆LV,
(SEQ ID NO: 17)
X₂RIVX₃RKX₄RFLX₅X₆LVR,
(SEQ ID NO: 18)
X₂RIVX₃RKX₄RFLX₅X₆LVRP,
(SEQ ID NO: 19)
X₂RIVX₃RKX₄RFLX₅X₆LVRPL,
(SEQ ID NO: 20)
X₂RIVX₃RKX₄RFLX₅X₆LVRPLR,
(SEQ ID NO: 21)
FX₂RIVX₃RKX₄RFLX₅X₆L,
(SEQ ID NO: 22)
FX₂RIVX₃RKX₄RFLX₅X₆LV,
(SEQ ID NO: 23)
FX₂RIVX₃RKX₄RFLX₅X₆LVR,
(SEQ ID NO: 24)
FX₂RIVX₃RKX₄RFLX₅X₆LVRP,
(SEQ ID NO: 25)
FX₂RIVX₃RKX₄RFLX₅X₆LVRPL,
(SEQ ID NO: 26)
FX₂RIVX₃RKX₄RFLX₅X₆LVRPLR,
(SEQ ID NO: 27)
EFX₂RIVX₃RKX₄RFLX₅X₆L,
(SEQ ID NO: 28)
EFX₂RIVX₃RKX₄RFLX₅X₆LV,
(SEQ ID NO: 29)
EFX₂RIVX₃RKX₄RFLX₅X₆LVR,
(SEQ ID NO: 30)
EFX₂RIVX₃RKX₄RFLX₅X₆LVRP,
(SEQ ID NO: 31)
EFX₂RIVX₃RKX₄RFLX₅X₆LVRPL,
(SEQ ID NO: 32)
EFX₂RIVX₃RKX₄RFLX₅X₆LVRPLR,
(SEQ ID NO: 33)
KEFX₂RIVX₃RKX₄RFLX₅X₆L,
(SEQ ID NO: 34)
KEFX₂RIVX₃RKX₄RFLX₅X₆LV,
(SEQ ID NO: 35)
KEFX₂RIVX₃RKX₄RFLX₅X₆LVR,
(SEQ ID NO: 36)
KEFX₂RIVX₃RKX₄RFLX₅X₆LVRP,
(SEQ ID NO: 37)
KEFX₂RIVX₃RKX₄RFLX₅X₆LVRPL,
(SEQ ID NO: 38)
KEFX₂RIVX₃RKX₄RFLX₅X₆LVRPLR,
(SEQ ID NO: 39)
GKEFX₂RIVX₃RKX₄RFLX₅X₆L,
(SEQ ID NO: 40)
GKEFX₂RIVX₃RKX₄RFLX₅X₆LV,
(SEQ ID NO: 41)
GKEFX₂RIVX₃RKX₄RFLX₅X₆LVR,
(SEQ ID NO: 42)
GKEFX₂RIVX₃RKX₄RFLX₅X₆LVRP,
(SEQ ID NO: 43)
GKEFX₂RIVX₃RKX₄RFLX₅X₆LVRPL,
(SEQ ID NO: 44)
GKEFX₂RIVX₃RKX₄RFLX₅X₆LVRPLR,
(SEQ ID NO: 45)
IGKEFX₂RIVX₃RKX₄RFLX₅X₆L,
(SEQ ID NO: 46)
IGKEFX₂RIVX₃RKX₄RFLX₅X₆LV,
(SEQ ID NO: 47)
IGKEFX₂RIVX₃RKX₄RFLX₅X₆LVR,
(SEQ ID NO: 48)
IGKEFX₂RIVX₃RKX₄RFLX₅X₆LVRP,
and
(SEQ ID NO: 49)
IGKEFX₂RIVX₃RKX₄RFLX₅X₆LVRPL,

with X₂-X₆ as defined above. Optionally, one amino acid in the sequence FX₂RIVX₃RKX₄RFLX₅X₆L (SEQ ID NO: 21) selected from the group of L, V, F, A, I, W, Y or Q, with the exception of X₂-X₆, is replaced by another amino acid selected from said group or by P.
In a further preferred embodiment, the fragment has the sequence X₂RIVX₃RKX₄RFLX₅X₆L (SEQ ID NO: 15), wherein:
X₂ is E, K, R, L, I, V, F, A, W, or V;
X₃ is E, K or R;
X₄ is E, R, L, I, V, F, A, W, or V;
X₅ is E, K, R, L, I, V, F, A, W, or V; and
X₆ is E, K or R.
   More specifically, the fragment has the sequence X₂RIVX₃RKX₄RFLX₅X₆L (SEQ ID NO: 15), wherein:
X₂ is K, L, I or V;
X₃ is E, K or R;
X₄ is L, I, V, F, A, W, or V;
X₅ is R, L, I or V; and
X₆ is E, K or R.
   Yet even more specifically, the fragment has the sequence X₂RIVX₃RKX₄RFLX₅X₆L (SEQ ID NO: 15), wherein:
X₂ is K or L;
X₃ is E or R;
X₄ is W;
X₅ is R or V; and
X₆ is E or K.

Preferably, the fragment peptide consists of any one of the above fragment sequences.

In a further preferred embodiment, the peptide is N-terminally modified, preferably with an acetyl, hexanoyl, decanoyl, myristoyl, PEGyl, propionyl or dye moiety, and/or C-terminally modified, preferably with an amide, PEGyl, amino-hexanoyl or dye moiety.

In another preferred embodiment, the peptide comprises an N-terminal acetyl moiety or a C-terminal amide moiety, preferably both.

It is particularly preferred that the length of the peptide is 14-50 amino acids, preferably 24-40 amino acids, in particular 24-30 amino acids.

One skilled in the art can easily synthesize the peptides of the present invention upon having read the present specification. Standard procedures for preparing synthetic peptides are well known in the art. Peptides of the present invention can be synthesized by commonly used methods as t-BOC or FMOC protection, preferably FMOC protection, of alpha-amino groups. Both methods involve stepwise syntheses whereby a single amino acid is added at each step starting from the carboxyl-terminus of the peptide (See, Coligan et al., Current Protocols in Immunology, Wiley Interscience, 1991, Unit 9). Peptides of the invention can also be synthesized by the solid phase peptide synthesis methods well known in the art. (Merrifield, J. Am. Chem. Soc., 85:2149, 1963), and Stewart and Young, Solid Phase Peptides Synthesis, Pierce, Rockford, Ill. (1984)). Peptides can be synthesized using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about 0.25 to 1 hour at 0° C. After evaporation of the reagents, the peptides are extracted from the polymer with 1% acetic acid solution which is then lyophilized to yield the crude material. This can typically be purified by such techniques as gel filtration on Sephadex G-15 using 5% acetic acid as a solvent, by high pressure liquid chromatography, and the like. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and assessed by the solid phase Edman degradation (see e.g Protein Purification, M. P. Deutscher, ed. Methods in Enzymology, Vol 182, Academic Press, 1990). Automated synthesis using FMOC solid phase synthetic methods can be achieved using an automated peptide synthesizer.

Of course, it is also possible to produce the peptides of the present invention using recombinant methods. The peptides can be produced in microorganisms such as bacteria, yeast or fungi, in eukaryotic cells such as mammalian or insect cells, or in a recombinant virus vector such as adenovirus, poxvirus, herpesvirus, Simliki forest virus, baculovirus, bacteriophage, sindbis virus or sendai virus. Suitable bacteria for producing the peptides include E. coli, B. subtilis or any other bacterium that is capable of expressing such peptides. Suitable yeast cells for expressing the peptides of the present invention include Saccharomyces cerevisiae, Schizosaccharomyces pombe, Candida, Pichia pastoris or any other yeast capable of expressing peptides. Corresponding means and methods are well known in the art. Also, methods for isolating and purifying recombinantly produced peptides are well known in the art and include e.g. gel filtration, affinity chromatography, ion exchange chromatography etc.

To facilitate isolation of said peptides, fusion polypeptides may be made wherein the peptides are translationally fused (covalently linked) to a heterologous polypeptide which enables isolation by affinity chromatography. Typical heterologous polypeptides are His-Tag (e.g. His6; 6 histidine residues), GST-Tag (Glutathione-S-transferase) etc. The fusion polypeptide facilitates not only the purification of the peptides but can also prevent the degradation of the peptides during the purification steps. If it is desired to remove the heterologous polypeptide after purification, the fusion polypeptide may comprise a cleavage site at the junction between the peptide and the heterologous polypeptide. The cleavage site may consist of an amino acid sequence that is cleaved with an enzyme specific for the amino acid sequence at the site (e.g. proteases).

The peptides of the present invention may form a salt by addition of an acid. Examples of the acid include inorganic acids (such as trifluoroacetic acid, hydrochloric acid, hydrobromic acid, phosphoric acid, nitric acid, and sulfuric acid) or organic carboxylic acids (such as acetic acid, propionic acid, maleic acid, succinic acid, malic acid, citric acid, tartaric acid, and salicylic acid), acidic sugars such as glucuronic acid, galacturonic acid, gluconic acid, ascorbic acid, etc., acidic polysaccharides such as hyaluronic acid, chondroitin sulfates, alginic acid, or organic sulfonic acids (such as methanesulfonic acid, and p-toluenesulfonic acid), and the like. Of these salts, preferred is a pharmaceutically acceptable salt.

The peptides of the present invention may form a salt with a basic substance. Examples of the salt include, for example, pharmaceutically acceptable salts selected from salts with inorganic bases such as alkali metal salts (sodium salt, lithium salt, potassium salt etc.), alkaline earth metal salts, ammonium salts, and the like or salts with organic bases, such as diethanolamine salts, cyclohexylamine salts and the like.

In the context of the present invention, the individual (to be treated with the peptide of the present invention) may be a human or non-human animal, preferably a non-human primate, a sheep, a pig, a dog or a rodent, in particular a mouse.

According to another preferred embodiment, the microbial infection (to be treated or prevented in the individual) is a bacterial infection or a fungal infection.

Preferably, the peptide of the invention is for prevention or treatment of a bacterial infection caused by Gram-positive bacteria such as Staphylococcus aureus, Streptococcus pyogenes, and Enterococcus faecalis, and/or Gram-negative bacteria such as Escherichia coli, Pseudomonas aeruginosa, and Klebsiella pneumoniae.

Fungal infections are caused by pathogenic fungi, which can be broadly classified into yeasts and molds. Examples of yeasts causing infections include Candida species, such as Candida albicans. Examples of molds that can cause infections are Aspergillus species, which can lead to conditions like aspergillosis, particularly in immunocompromised individuals. According to a further preference, the peptide is for prevention or treatment of an infection caused by pathogenic yeast, such as pathogens of the genus Candida, or pathogenic mold, such as pathogens of the genus Aspergillus.

In another preferred embodiment, the peptide is administered in combination with at least one antibiotic. The antibiotic may be selected from various conventional classes of antibiotics. The combination therapy may have synergistic effects, enhancing the efficacy of both the peptide and the antibiotic, while reducing the risk of resistance development. Preferred classes of antibiotics for combination with the peptide include the following:

Beta-lactams: These antibiotics inhibit cell wall synthesis in bacteria. Especially preferred antibiotics within this class include penicillins (e.g. amoxicillin and ampicillin), cephalosporins (e.g. cefazolin, ceftriaxone and ceftazidime), carbapenems (e.g. imipenem, meropenem and ertapenem), and monobactams (e.g. aztreonam).

Tetracyclines: This class of antibiotics targets protein synthesis by binding to the bacterial ribosome. Especially preferred are doxycycline, minocycline, and tigecycline.

Macrolides: These antibiotics also inhibit protein synthesis by interacting with the bacterial ribosome. Erythromycin, clarithromycin, and azithromycin are especially preferred.

Aminoglycosides: Aminoglycoside antibiotics disrupt bacterial protein synthesis by binding to the ribosome. Particularly preferred are gentamicin, amikacin, and tobramycin.

Fluoroquinolones: This class of antibiotics interferes with bacterial DNA replication by inhibiting DNA gyrase and topoisomerase IV enzymes. Preferred fluoroquinolones are ciprofloxacin, levofloxacin, and moxifloxacin.

Intravenous administration of the peptide is preferred. However, other modes of administration are also possible.

The peptides of the present invention are preferably administered to the individual in need thereof in an amount of 100 μg/kg body weight to 100 mg/kg body weight, preferably 1 mg/kg body weight to 50 mg/kg body weight, more preferably 5 mg/kg body weight to 15 mg/kg body weight, in particular 10 mg/kg body weight. Alternatively, or in addition thereto, the peptides of the present invention are preferably administered daily (e.g. three times a day, twice a day or once a day), every 2nd, every 3rd, every 4^{th}, every 5th day, every 6th day or weekly.

The peptide of the present invention may be provided in a pharmaceutical composition. This pharmaceutical composition is preferably provided with at least one excipient. Excipients suitable for the pharmaceutical composition of the present invention are known to the person skilled in the art, upon having read the present specification, for example water (especially water for injection), saline, Ringer's solution, dextrose solution, buffers, Hank solution, 5% dextrose in saline, substances that enhance isotonicity and chemical stability, buffers and preservatives. This pharmaceutical composition can (as a drug) be administered via appropriate procedures known to the skilled person (upon having read the present specification) to a patient or individual in need thereof (i.e. a patient or individual having or having the risk of developing the diseases or conditions mentioned herein). The preferred route of administration of said pharmaceutical composition is parenteral administration, in particular through intraperitoneal, subcutaneous, intramuscular and/or intravenous administration. The dosage and method of administration depends on the individual patient or individual to be treated. Said pharmaceutical composition can be administered in any suitable dosage known from other biological dosage regimens or specifically evaluated and optimised for a given individual. For example, the nucleic acid cargo may be present in the pharmaceutical composition in an amount from 1 mg to 10 g, preferably 50 mg to 2 g, in particular 100 mg to 1 g. Usual dosages can also be determined on the basis of kg body weight of the patient, for example preferred dosages are in the range of 0.1 mg to 100 mg/kg body weight, especially 1 to 10 mg/kg body weight (per administration session). The administration may occur e.g. once daily, once every other day, once per week or once every two weeks. As the preferred mode of administration of the inventive pharmaceutical composition is parenteral administration, the pharmaceutical composition according to the present invention is preferably liquid or ready to be dissolved in liquid such sterile, de-ionised or distilled water or sterile isotonic phosphate-buffered saline (PBS). Preferably, 1000 μg (dry-weight) of such a composition comprises 0.1-990 μg, preferably 1-900 μg, more preferably 10-200 μg compound, and option-ally 1-500 μg, preferably 1-100 μg, more preferably 5-15 μg (buffer) salts (preferably to yield an isotonic buffer in the final volume), and optionally 0.1-999.9 μg, preferably 100-999.9 μg, more preferably 200-999 μg other excipients. Preferably, 100 mg of such a dry composition is dissolved in sterile, de-ionised/distilled water or sterile isotonic phosphate-buffered saline (PBS) to yield a final volume of 0.1-100 ml, preferably 0.5-20 ml, more preferably 1-10 ml.

The term “preventing” or “prevention” as used herein means to stop a disease state or condition from occurring in a patient or subject completely or almost completely or at least to a (preferably significant) extent, especially when the patient or subject or individual is predisposed to such a risk of contracting a disease state or condition.

The peptide of the invention exhibits antimicrobial activity, preferably antibacterial and/or antifungal activity, with a particular preference for antibacterial and/or antifungal activities. Additionally, the peptide may exhibit anti-inflammatory properties.

Herein, the expression “antimicrobial activity” or “antimicrobial” in relation to the peptide describes the capacity to combat the growth or proliferation of at least one microbe, such as a bacterium or a fungus. This capacity may include the inhibition and reduction of microbial growth, as well as the lysis of the microbes.

Antimicrobial activity can be expressed as inhibitory concentration (IC) or lethal concentration (LC). The LCₓ refer to the minimum peptide concentration that kills at least x % of microbes after 2 hours (at the optimum growth temperature of the microbes, e.g. 37° C.). For example, LC_{99.9%} indicates the lowest peptide concentration that kills more than 99.9% of microbes. Antimicrobial, antibacterial, antiviral, antifungal, and antiparasitic activities can be measured using established methods in the field.

Preferably, the inventive peptide has a LC_{99.9%} of less than 50 μM, preferably less than 25 μM, more preferably less than 10 μM, even more preferably less than 5 μM against a microbe such as any one of the microorganisms mentioned herein.

In the context of the present invention, the test whether a peptide has a hemolytic activity which is lower than the hemolytic activity of peptide OP-145 (acetyl-IGKEFKRIVERIKRFLRELVRPLR-amide (SEQ ID NO: 50)) may be performed according to any method known in the art, as for instance disclosed in Avrahami & Shai, 2004. In particular, a 0.5% erythrocyte solution in phosphate-buffered saline is exposed to the respective peptide (e.g. at 1 μM or 10 μM or 20 μM or 50 μM) at 37° C. for 1 h; hemolysis is determined by measuring optical density of the cell supernatants at 415 nm. More specifically, the assay may be performed as follows: Whole venous blood of healthy volunteers is collected in citrate tubes. Erythrocytes are isolated by centrifuging the blood for 10 min at 2,000×g and subsequently washing twice in PBS. A 0.5% erythrocyte solution in PBS is exposed to the peptides, e.g. at 1 μM or 10 μM or 20 μM or 50 μM. After 1 h at 37° C. under static conditions, the erythrocyte suspensions are centrifuged and the optical density of the supernatants is determined at 415 nm. The percentage of hemolysis may be calculated relative to a positive control (e.g. 1% Triton X-100).

In the context of the present invention, the test whether a peptide has an antimicrobial activity which is higher than the antimicrobial activity of peptide OP-145 (acetyl-IGKEFKRIVERIKRFLRELVRPLR-amide (SEQ ID NO: 50)) may be performed according to any method known in the art, as for instance disclosed in Balouiri et al, 2016. In particular, S. aureus JAR060131, Enterococcus hirae ATCC 10541, Bacillus subitlis 168C trpC2 or Escherichia coli ATCC25992 are cultured to mid-logarithmic phase in tryptic soy broth, brain heart infusion (BHIB), mueller hinton (MHB) or luria broth (LB) and washed once with PBS. Approximately 1×10⁶ CFU/ml of PBS are incubated with the respective peptide (final concentrations: 0-102.4 μM) for 0-2 h at 37° C. under shaking conditions. Thereafter, the number of viable bacteria is determined by plating on diagnostic sensitivity agar. Antimicrobial activity is expressed as the 99.9% killing concentration (LC_{99.9}), i.e., the lowest peptide concentration that killed ≥99.9% of bacteria. An antimicrobial activity being e.g., 10% higher than a reference antimicrobial activity means that the LC_{99.9} is 10% lower than the respective reference antimicrobial activity.

The present invention further relates to the following embodiments:

Embodiment 1. An antimicrobial peptide comprising the sequence IGKX₁FX₂RIVX₃RKX₄RFLX₅X₆LVRPLX₇ (SEQ ID NO: 7), wherein each of X₁, X₂, X₃, X₄, X₅, X₆, and X₇ is an amino acid independently selected from the group consisting of E, K, R, L, I, V, F, A, W, V and P, preferably from the group consisting of E, K, R, L, I, V, F, A, W and V, more preferably from the group consisting of E, K, R, L, W and V; optionally wherein one amino acid in the sequence IGKX₁FX₂RIVX₃RKX₄RFLX₅X₆LVRPLX₇ (SEQ ID NO: 7) selected from the group of L, V, F, A, I, W, Y or Q, with the exception of X₁-X₇, is replaced by another amino acid selected from said group or by P.

Embodiment 2. The peptide of embodiment 1, wherein:
X₁ is E, K or R;
X₂ is E, K, R, L, I, V, F, A, W, or V;
X₃ is E, K or R;
X₄ is E, R, L, I, V, F, A, W, or V;
X₅ is E, K, R, L, I, V, F, A, W, or V;
X₆ is E, K or R; and
X₇ is E, K, R, L, I, V, F, A, W, or V.

Embodiment 3. The peptide of embodiment 2, wherein:
X₁ is E, K or R;
X₂ is K, L, I or V;
X₃ is E, K or R;
X₄ is L, I, V, F, A, W, or V;
X₅ is R, L, I or V;
X₆ is E, K or R; and
X₇ is R, L, I or V.

Embodiment 4. The peptide of embodiment 3, wherein:
X₁ is E or K;
X₂ is K or L;
X₃ is E or R;
X₄ is W;
X₅ is R or V;
X₆ is E or K; and
X₇ is R or L.

Embodiment 5. The peptide of any one of embodiments 1 to 4, wherein the peptide has a sequence selected from the group consisting of IGKEFKRIVERKWRFLRELVRPLR (SEQ ID NO: 2), IGKKFKRIVRRKKRFLRKLVRPLR (SEQ ID NO: 3), IGKEFKRIVERKWRFLRKLVRPLR (SEQ ID NO: 4), IGKEFLRIVERKWRFLRKLVRPLL (SEQ ID NO: 5) and IGKEFLRIVERKWRFLVKLVRPLL (SEQ ID NO: 6).

Embodiment 6. The peptide of any one of embodiments 1 to 5, wherein the entire amino acid sequence of the peptide consists of said sequence.

Embodiment 7. An antimicrobial peptide which is a fragment of the peptide of any one of embodiments 1 to 6, wherein the fragment has the sequence X₂RIVX₃RKX₄RFLX₅ (SEQ ID NO: 51), preferably X₂RIVX₃RKX₄RFLX₅X₆L (SEQ ID NO: 14), wherein each of X₂, X₃, X₄, X₅ and X₆ is an amino acid independently selected from the group consisting of E, K, R, L, I, V, F, A, W, V and P, preferably from the group consisting of E, K, R, L, I, V, F, A, W and V, more preferably from the group consisting of E, K, R, L, W and V;
optionally wherein one amino acid in the sequence X₂RIVX₃RKX₄RFLX₅X₆L (SEQ ID NO: 14) selected from the group of L, V, F, A, I, W, Y or Q, with the exception of X₂-X₆, is replaced by another amino acid selected from said group or by P.

Embodiment 8. The peptide of embodiment 7, wherein the fragment has the sequence X₂RIVX₃RKX₄RFLX₅X₆L (SEQ ID NO: 14), wherein:
X₂ is E, K, R, L, I, V, F, A, W, or V;
X₃ is E, K or R;
X₄ is E, R, L, I, V, F, A, W, or V;
X₅ is E, K, R, L, I, V, F, A, W, or V; and
X₆ is E, K or R.

Embodiment 9. The peptide of embodiment 8, wherein the fragment has the sequence X₂RIVX₃RKX₄RFLX₅X₆L (SEQ ID NO: 14), wherein:
X₂ is K, L, I or V;
X₃ is E, K or R;
X₄ is L, I, V, F, A, W, or V;
X₅ is R, L, I or V; and
X₆ is E, K or R.

Embodiment 10. The peptide of embodiment 9, wherein the fragment has the sequence X₂RIVX₃RKX₄RFLX₅X₆L (SEQ ID NO: 14), wherein:
X₂ is K or L;
X₃ is E or R;
X₄ is W;
X₅ is R or V; and
X₆ is E or K.

Embodiment 11. The peptide of any one of embodiments 7 to 10, wherein the fragment has a sequence selected from the group consisting of KRIVERKWRFLREL (SEQ ID NO: 53), KRIVRRKKRFLRKL (SEQ ID NO: 54), KRIVERKWRFLRKL (SEQ ID NO: 55), LRIVERKWRFLRKL (SEQ ID NO: 56) and LRIVERKWRFLVKL (SEQ ID NO: 57).

Embodiment 12. The peptide which is a fragment according to any one of embodiments 7 to 11, wherein the length of the peptide is 12-23 amino acids, preferably 13-22 amino acids or even 14-21 amino acids, more preferably 14-20 amino acids or even 14-19 amino acids, even more preferably 14-18 amino acids or even 14-17 amino acids, yet even more preferably 14-16 amino acids or even 14-15 amino acids, especially 14 amino acids.

Embodiment 13. The peptide of any one of embodiments 1 to 12, wherein the peptide is N-terminally modified, preferably with an acetyl, hexanoyl, decanoyl, myristoyl, PEGyl, propionyl or dye moiety, and/or C-terminally modified, preferably with an amide, PEGyl, amino-hexanoyl or dye moiety.

Embodiment 14. The peptide of any one of embodiments 1 to 13, wherein the peptide comprises an N-terminal acetyl moiety and/or a C-terminal amide moiety.

Embodiment 15. The peptide of any one of embodiments 1 to 14, wherein the length of the peptide is 14-50 amino acids, preferably 24-40 amino acids, in particular 24-30 amino acids.

Embodiment 16. The peptide of any one of embodiments 1 to 15, wherein the peptide has a hemolytic activity which is lower than the hemolytic activity of peptide OP-145 (acetyl-IGKEFKRIVERIKRFLRELVRPLR-amide (SEQ ID NO: 50)), preferably at least 10% lower, more preferably at least 20% lower, even more preferably at least 30% lower, yet even more preferably at least 40% lower, especially at least 50% lower.

Embodiment 17. The peptide of any one of embodiments 1 to 16, wherein the peptide has an antimicrobial activity which is higher than the antimicrobial activity of peptide OP-145 (acetyl-IGKEFKRIVERIKRFLRELVRPLR-amide (SEQ ID NO: 50)), preferably at least 10% higher, more preferably at least 20% higher, even more preferably at least 30% higher, yet even more preferably at least 40% higher, especially at least 50% higher.

Embodiment 18. The peptide of any one of embodiments 1 to 17, for use in therapy, preferably antimicrobial therapy.

Embodiment 19. The peptide of any one of embodiments 1 to 18, for use in the prevention or treatment of a microbial infection, preferably wherein the peptide is administered in combination with at least one antibiotic.

Embodiment 20. The peptide for use according to embodiment 18 or 19, wherein the peptide is administered intravenously.

Embodiment 21. A method, comprising
obtaining a pharmaceutical composition comprising the peptide of any one of embodiments 1 to 20; and
   administering the pharmaceutical composition to an individual, wherein the individual has a microbial infection or is at risk of developing a microbial infection.

The present invention is further illustrated by the following figures and examples, without being restricted thereto.

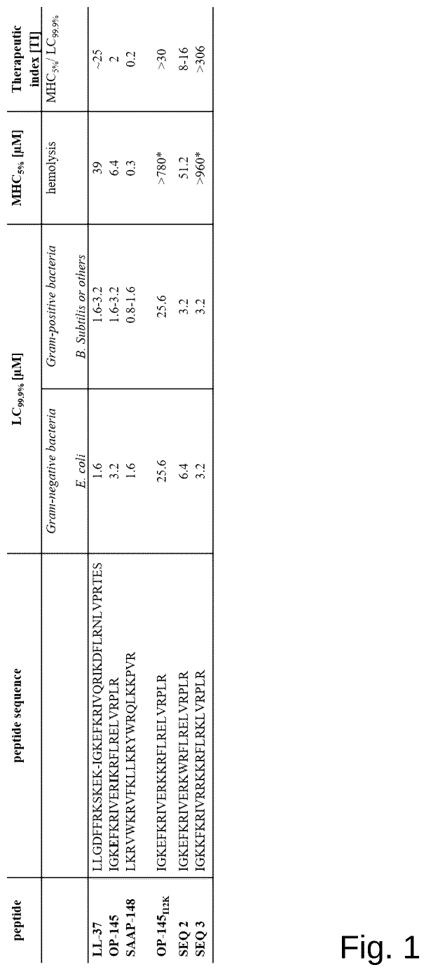

FIG. 1—Improved antimicrobial activity. The inventive peptides with the SEQ ID NO: 2 and 3 show an improved antimicrobial activity compared to OP-145_{I12K} (the peptide they were derived from). The peptide with SEQ ID NO: 3 also shows a much improved therapeutic index. * not hemolytic (higher concentrations were not tested).

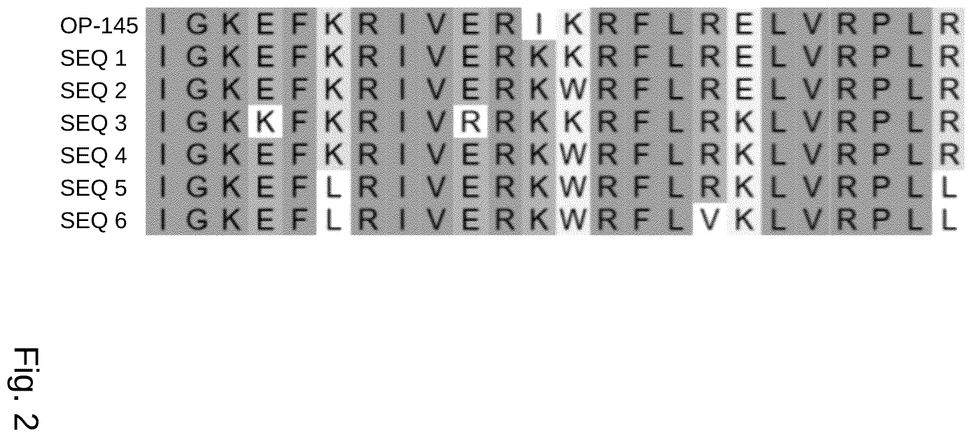

FIG. 2—Sequence alignments. Alignment of parent peptide OP-145, peptide OP-145_{I12K} (SEQ ID NO: 1) and derived sequences SEQ ID NOs: 2-6 of inventive peptides.

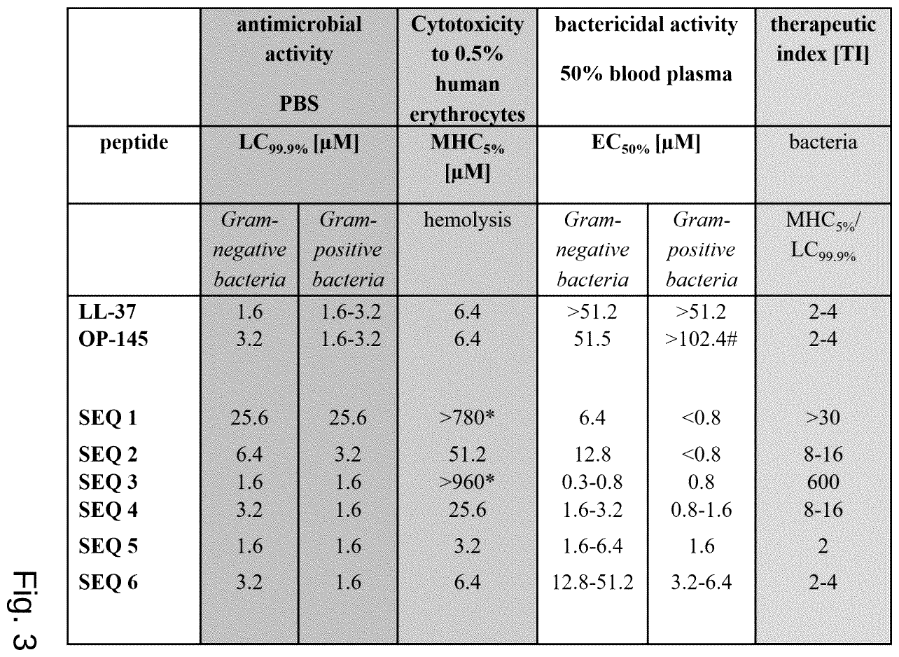

FIG. 3—Improved antimicrobial activity and therapeutic index. Activities of peptides with SEQ ID NOs: 1-6 compared to activities of known peptides LL-37, OP-145 (derivative of LL-37) and SAAP-148 (derivative of OP-145). Activities tested are antimicrobial and hemolytic activities (for 1 h treatment with peptides) according to de Breij et al., 2018, and Malanovic et al., 2015, for 1 hour treatment of bacterial and human cells. Antimicrobial activity is expressed as LC_{99.9%}—this is the lethal concentration causing death in 99.9% cells, hemolytic activity as MHC5% showing the minimal hemolytic concentration generally tolerated by FDA (=5% hemolysis). *not hemolytic (higher concentrations were not tested). Results are ranges of at least three independent experiments. If no range is given, then LC_{99.9} values were identical in all experiments. #data observed by de Breij et al., 2018.

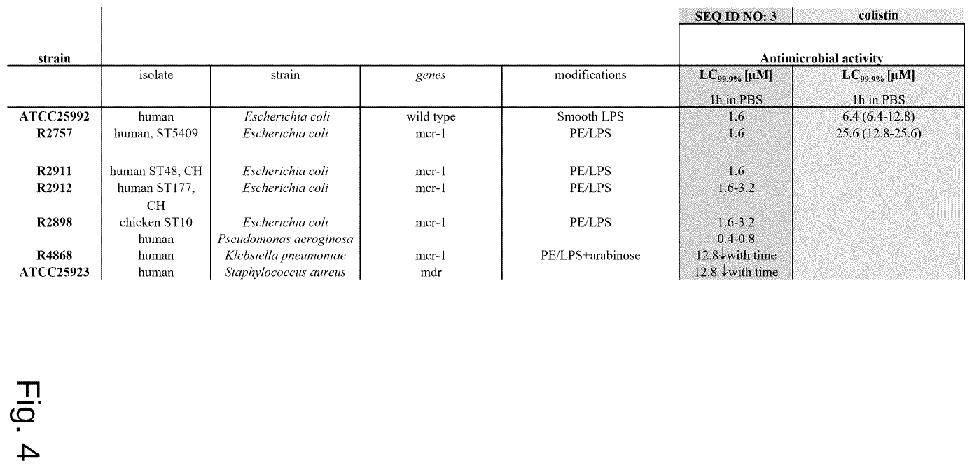

FIG. 4—Excellent antimicrobial activity of peptide SEQ ID NO: 3 against multi-resistant clinical isolates. Bactericidal activity was tested (for 1 h treatment with peptides) in presence of PBS according to de Breij et al., 2018. Bactericidal activity is expressed as LC_{99.9%}—is the lethal concentration causing death in 99.9% cells. Results are ranges of at least three independent experiments. If no range is given, then LC_{99.9} values were identical in all experiments.

### EXAMPLE 1—ANTIMICROBIAL PROPERTIES OF THE INVENTIVE PEPTIDES

### Materials & Methods

Peptides: The peptides prepared by solid phase strategies on an automated multiple peptide synthesizer (SyroII, MultiSyntech, Witten, Germany) as described previously (Hiemstra et al, 1997). The purity of the peptides was >95%, as determined by UPLC-MS (Acquity, Waters, Milford, Ma). Peptide integrity was confirmed using Maldi-Tof mass spectrometry (Microflex, Bruker, Bremen, Germany), showing the expected molecular masses. The lyophilized peptides were stored at −20° C. until use, then dissolved in H₂O with 0.01% acetic acid to a stock of 10 mg/ml, and aliquots were stored at −20° C.

Antimicrobial activity: S. aureus JAR060131, Enterococcus hirae ATCC 10541, Bacillus subitlis 168C trpC2, Escherichia coli ATCC25992 or their variants were cultured to mid-logarithmic phase in tryptic soy broth, brain heart infusion (BHIB), mueller hinton (MHB) or luria broth (LB) and washed once with PBS. Approximately 1×10⁶ CFU/ml of PBS were incubated with peptide (final concentrations: 0-102.4 μM) for 0-2 h at 37° C. under shaking conditions. Thereafter, the number of viable bacteria was determined by plating on diagnostic sensitivity agar. Antimicrobial activity is expressed as the 99.9% killing concentration (LC_{99.9}), i.e., the lowest peptide concentration that killed ≥99.9% of bacteria.

Hemolysis assay: Whole venous blood of healthy volunteers was collected in citrate tubes. Erythrocytes were isolated by centrifuging the blood for 10 min at 2,000×g and subsequently washed twice in PBS. A 0.5% erythrocyte solution in PBS was exposed to the peptides at the respective concentrations. After 1 h at 37° C. under static conditions, the erythrocyte suspensions were centrifuged and the optical density of the supernatants was determined at 415 nm. The percentage of hemolysis was calculated relative to the positive control (1% Triton X-100).

### Results

The inventive peptides IGKEFKRIVERKWRFLRELVRPLR and IGKKFKRIVRRKKRFLRKLVRPLR (SEQ ID NO: 2 and 3) showed an improved antimicrobial activity compared to OP-145_{I12K} (the peptide they were derived from, SEQ ID NO: 1), against both Gram-negative and Gram-positive bacteria, as shown in Table 1 below:

| | LC_{99.9%} [μM] | |
|---|---|---|
| | Gram-negative bacteria | Gram-positive bacteria |
| peptide | E. coli ATCC25992 | B. subtilis 168 trpC2 |
| SEQ ID NO: 1 | 25.6 | 25.6 |
| SEQ ID NO: 2 | 6.4 | 3.2 |
| SEQ ID NO: 3 | 3.2 | 3.2 |

This improved antimicrobial profile is expected for further I12K variants described herein.

In comparison to parent peptide OP-145, OP-145_{112K} and the variant peptides with the SEQ ID NO: 2 and 3 did not lyse the human erythrocytes. This beneficial behavior is expected for further I12K variants described herein (see also the alignment in FIG. 2 which underscores their similarity).

### EXAMPLE 2—FURTHER RESULTS

Further positive results related to the inventive peptides were obtained, see also FIGS. 3 and 4. The experimental details are similar to the details of Example 1, unless stated otherwise.

### NON-PATENT REFERENCES

Avrahami & Shai. “A new group of antifungal and antibacterial lipopeptides derived from non-membrane active peptides conjugated to palmitic acid.” Journal of Biological Chemistry 279.13 (2004): 12277-12285.
Balouiri, Mounyr, Moulay Sadiki, and Saad Koraichi Ibnsouda. “Methods for in vitro evaluating antimicrobial activity: A review.” Journal of pharmaceutical analysis 6.2 (2016): 71-79.
de Breij, A., et al. “Prevention of Staphylococcus aureus biomaterial-associated infections using a polymer-lipid coating containing the antimicrobial peptide OP-145.” Journal of Controlled Release 222 (2016): 1-8.
de Breij, Anna, et al. “The antimicrobial peptide SAAP-148 combats drug-resistant bacteria and biofilms.” Science translational medicine 10.423 (2018): eaan4044.
Chen, Keqiang, et al. “The potentials of short fragments of human anti-microbial peptide LL-37 as a novel therapeutic modality for diseases.” Frontiers in Bioscience-Landmark 26.11 (2021): 1362-1372.
Haisma, Elisabeth M., et al. “LL-37-derived peptides eradicate multidrug-resistant Staphylococcus aureus from thermally wounded human skin equivalents.” Antimicrobial agents and chemotherapy 58.8 (2014): 4411-4419.
Ön, Ayse. “Membrane permeabilization of Gram-negative bacteria by antimicrobial peptides derived from the human cathelicidin LL-37.” (2017), Graz University of Technology, master thesis.
Malanovic, Nermina, et al. “Phospholipid-driven differences determine the action of the synthetic antimicrobial peptide OP-145 on Gram-positive bacterial and mammalian membrane model systems.” Biochimica et Biophysica Acta (BBA)-Biomembranes 1848.10 (2015): 2437-2447.
Nell, Marja J., et al. “Development of novel LL-37 derived antimicrobial peptides with LPS and LTA neutralizing and antimicrobial activities for therapeutic application.” Peptides 27.4 (2006): 649-660.
Piller, Paulina. “Mode of action of LL-37 derived antimicrobial peptides in Enterococcus hirae.” (2019), Graz University of Technology, master thesis.

1. An antimicrobial peptide comprising the sequence IGKX₁FX₂RIVX₃RKX₄RFLX₅X₆LVRPLX₇ (SEQ ID NO: 7), wherein each of X₁, X₂, X₃, X₄, X₅, X₆, and X₇ is an amino acid independently selected from the group consisting of E, K, R, L, I, V, F, A, W, V and P, preferably from the group consisting of E, K, R, L, I, V, F, A, W and V, more preferably from the group consisting of E, K, R, L, W and V; optionally wherein one amino acid in the sequence IGKX₁FX₂RIVX₃RKX₄RFLX₅X₆LVRPLX₇ (SEQ ID NO: 7) selected from the group of L, V, F, A, I, W, Y or Q, with the exception of X₁-X₇, is replaced by another amino acid selected from said group or by P.

2. The peptide of claim 1, wherein:
X₁ is E or K;
X₂ is K or L;
X₃ is E or R;
X₄ is W;
X₅ is R or V;
X₆ is E or K; and
X₇ is R or L.

3. The peptide of claim 1, wherein the peptide has a sequence selected from the group consisting of
(SEQ ID NO: 2)
IGKEFKRIVERKWRFLRELVRPLR,
(SEQ ID NO: 3)
IGKKFKRIVRRKKRFLRKLVRPLR,
(SEQ ID NO: 4)
IGKEFKRIVERKWRFLRKLVRPLR,
(SEQ ID NO: 5)
IGKEFLRIVERKWRFLRKLVRPLL
and
(SEQ ID NO: 6)
IGKEFLRIVERKWRFLVKLVRPLL

4. The peptide of claim 1, wherein the entire amino acid sequence of the peptide consists of said sequence.

5. An antimicrobial peptide which is a fragment of the peptide of claim 1, wherein the fragment has the sequence X₂RIVX₃RKX₄RFLX₅X₆L (SEQ ID NO: 14), wherein each of X₂, X₃, X₄, X₅ and X₆ is an amino acid independently selected from the group consisting of E, K, R, L, I, V, F, A, W, V and P, preferably from the group consisting of E, K, R, L, I, V, F, A, W and V, more preferably from the group consisting of E, K, R, L, W and V; optionally wherein one amino acid in the sequence X₂RIVX₃RKX₄RFLX₅X₆L (SEQ ID NO: 14) selected from the group of L, V, F, A, I, W, Y or Q, with the exception of X₂-X₆, is replaced by another amino acid selected from said group or by P.

6. The peptide of claim 5, wherein the fragment has the sequence X₂RIVX₃RKX₄RFLX₅X₆L (SEQ ID NO: 15), wherein:
X₂ is K or L;
X₃ is E or R;
X₄ is W;
X₅ is R or V; and
X₆ is E or K.

7. The peptide of claim 5, wherein the fragment has a sequence selected from the group consisting of
(SEQ ID NO: 53)
KRIVERKWRFLREL,
(SEQ ID NO: 54)
KRIVRRKKRFLRKL,
(SEQ ID NO: 55)
KRIVERKWRFLRKL,
(SEQ ID NO: 56)
LRIVERKWRFLRKL
and
(SEQ ID NO: 57)
LRIVERKWRFLVKL

8. The peptide which is a fragment according to claim 5, wherein the length of the peptide is 12-23 amino acids, preferably 13-22 amino acids or even 14-21 amino acids, more preferably 14-20 amino acids or even 14-19 amino acids, even more preferably 14-18 amino acids or even 14-17 amino acids, yet even more preferably 14-16 amino acids or even 14-15 amino acids, especially 14 amino acids.

9. The peptide of claim 1, for use in therapy, preferably antimicrobial therapy.

10. The peptide of claim 1, for use in the prevention or treatment of a microbial infection, preferably wherein the peptide is administered in combination with at least one antibiotic.
